(19) United States
(12) Patent Application Publication
(10) Pub. No.: US 2010/0318106 A1
(43) Pub. Date: Dec. 16, 2010

(54) SUTURE LOOP ISOMETER

(75) Inventor: Scott Trenhaile, Belvidere, IL (US)

Correspondence Address: DICKE, BILLIG & CZAJA, FIFTH STREET TOWERS, 100 SOUTH FIFTH STREET, SUITE 2250, MINNEAPOLIS, MN, 55402, US

(73) Assignee: Rockford Orthopaedic Sports Medicine Services, LLC, Belvidere, IL (US)

(21) Appl. No.: 12/782,366

(22) Filed: May 18, 2010

Related U.S. Application Data
(60) Provisional application No. 61/179,120, filed on May 18, 2009.

(51) Int. Cl. A61B 17/04

(52) U.S. Cl. 606/148

(57) ABSTRACT

A suture loop isometer includes an elongated body; a first loop provided at one end of the elongated body; a T-bar provided at an opposite end of the elongated body; at least one leg extended from one end of the T-bar; and a second loop provided at an end of the at least one leg.

### CROSS REFERENCE TO RELATED APPLICATIONS

This application claims priority under 35 U.S.C. §119(e) to U.S. Provisional Patent Application Ser. No. 61/179,120 filed on May 18, 2009, and incorporated herein by reference.

### FIELD OF THE INVENTION

The present invention relates generally to a medical device that facilitates orthopaedic procedures when reconstructing intra-articular ligaments in a knee including anterior cruciate ligament (ACL) and posterior cruciate ligament (PCL) reconstructions. More specifically, the device provides a system including a suture loop and a double slotted guide pin, wherein the device would allow for identifying an isometric point of a proposed ligament reconstruction prior to drilling large sockets in the bone for the ligament reconstruction.

### BACKGROUND

ACL reconstruction is one of the most commonly performed procedures on the human knee. Primary surgical goals include restoring translational and rotational stability of the knee utilizing a soft tissue graft that is fixated on the femoral and tibial sides of the joint. The most common cause of graft failure is technical error. This commonly occurs with incorrect placement of a femoral and/or tibial tunnel. An ideal tunnel placement for a graft is one in which the graft remains isometric throughout a full range of motion. In ACL surgery, the femoral tunnel is typically fixated first with various means of fixation. The knee is then cycled numerous times while holding tension manually on the tibial side of the graft. If the graft is not isometric, pistoning of the graft within the tunnel occurs due to a cam effect of the femur in relation to the tibia. At this point in the procedure, the femoral and tibial tunnels are already drilled and the graft is in place. Correcting a non-isometric graft is a major undertaking at this point in the procedure and may not be possible.

ACL surgery may be performed utilizing various techniques include a trans-tibial approach, medial portal approach, and double bundle reconstructions. Multi-ligamentous reconstructions (ACL and PCL) may also be required.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates one embodiment of a suture loop isometer.

FIG. 2 illustrates one embodiment of a guide pin.

FIGS. 3-6b illustrate embodiments of a trans-tibial ACL approach according utilizing the suture loop isometer of FIG. 1 and guide pin of FIG. 2.

FIGS. 7-13 illustrate embodiments of a medial portal approach.

FIGS. 14-22 illustrate embodiments of a double bundle approach.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

As described herein, one goal of this system is to assess isometricity of an ACL graft prior to committing to tunnel drilling and to allow for adjustments. With more complex reconstructions, like a double bundle ACL, ligament footprint attachment size may be assessed for feasibility of the procedure. The device described herein provides more accuracy to ACL and PCL reconstruction procedures.

### Device

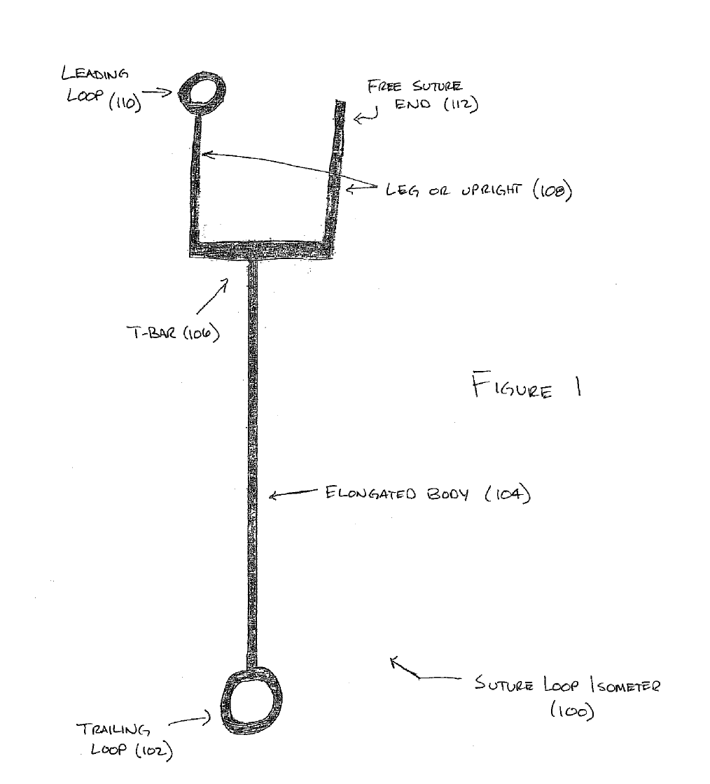

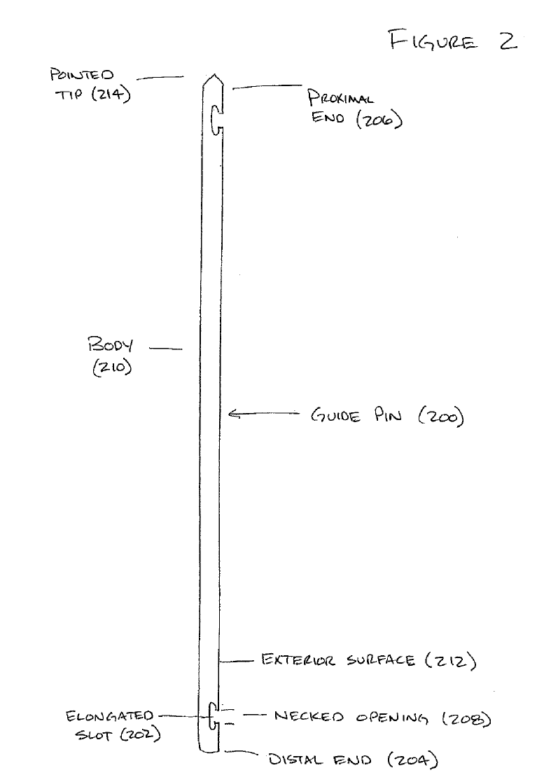

FIG. 1 illustrates one embodiment of a suture loop isometer according to the present invention. The suture loop isometer device of FIG. 1 includes a trailing loop attached at one end of an elongated body and a “T-bar” at the opposing end of the elongated body. In one embodiment, the T-bar extends perpendicular to the elongated body. The T-bar is available in various lengths, for example, 5 mm, 6 mm, 7 mm or other suitable length. In one embodiment, uprights or legs extend from each end of the T-bar. In one embodiment, a leading loop and a free end extend from the respective legs of the T-bar. In one embodiment, the leading loop and the free end extend from the T-bar generally parallel to each other in a direction opposite of the elongated body. In one embodiment, the suture loop isometer is a flexible material. In one embodiment, the T-bar section is of a material of lesser flexibility than the rest of the suture loop isometer. In one embodiment, the suture loop isometer is an absorbable suture material.

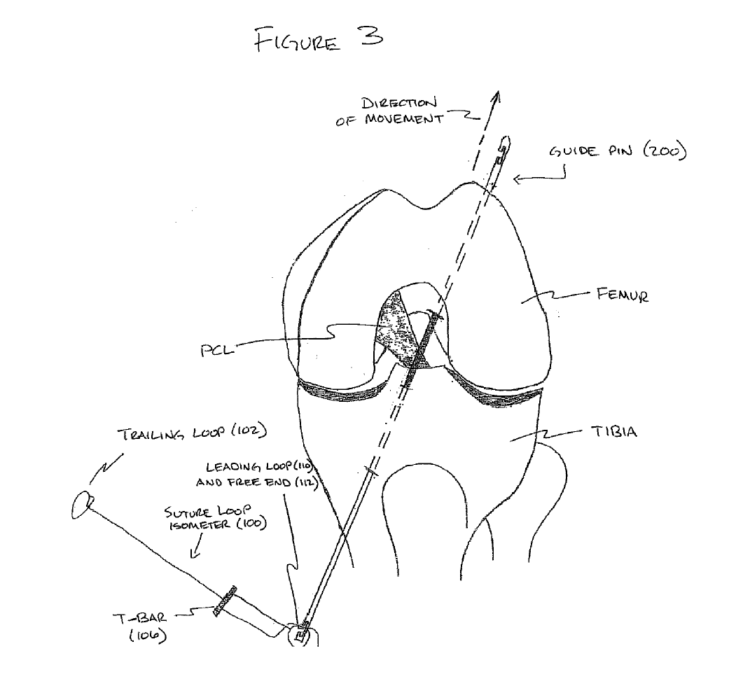

FIG. 2 illustrates one embodiment of a double slotted guide pin according to the present invention. In one embodiment, the guide pin includes elongated slots at each of the opposite ends of the guide pin. In one embodiment, the elongated slots are inset into the guide pin a dimension which provides a “necked” opening, extending from the elongated slot to an exterior surface of the guide pin. The guide pin includes a distal end and a proximal end. In one embodiment, the guide pin is pointed on both the distal and proximal ends. In another embodiment, the guide pin is pointed on one end. In one embodiment, the body of the guide pin is constructed of K-wire, a smooth pin, stainless steel rod, or suitable material. In one embodiment, the diameter of the guide pin is 2.4 mm, for example, although other diameters may also be suitable.

### Techniques

### A. Trans-Tibial ACL Approach

When performing an arthroscopic-assisted ACL reconstruction using the standard trans-tibial approach, the tibial hole is drilled first from inferior and medial on the tibia in a retrograde fashion. Once the guide pin is placed in a satisfactory position, a reamer is passed over the guide pin to create the tibial hole. The guide pin is then placed through the tibial hole, using a standard ACL guide, across the joint, and into the femur (while the knee is in a flexed position). A femoral reamer is then passed through the tibia, across the joint, and into the femur over the guide pin to a superior and lateral position.

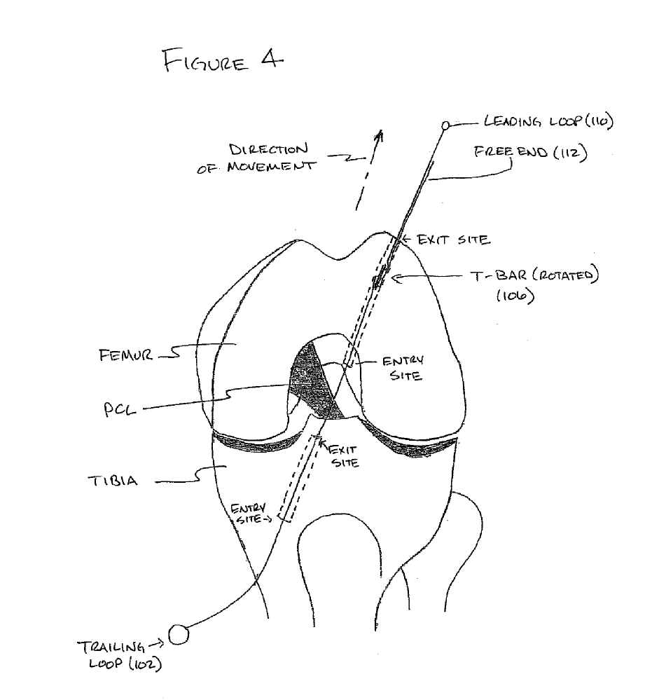

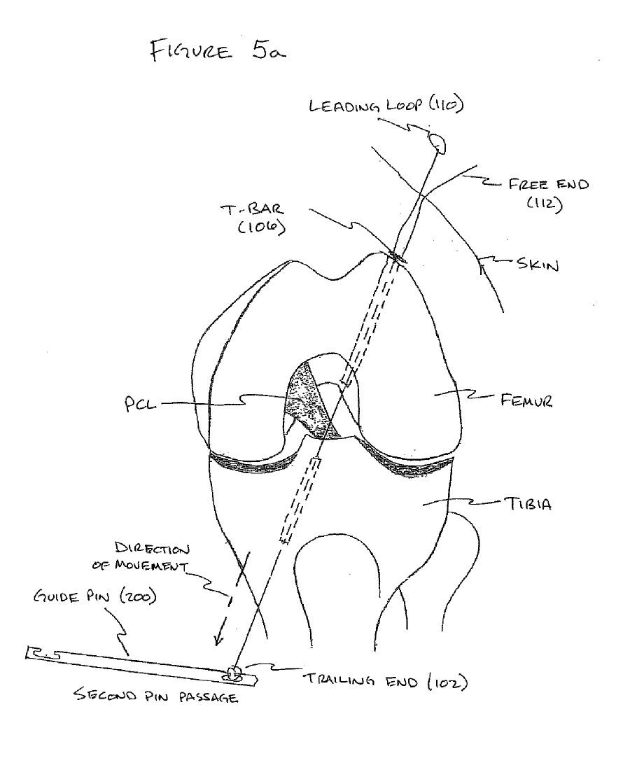

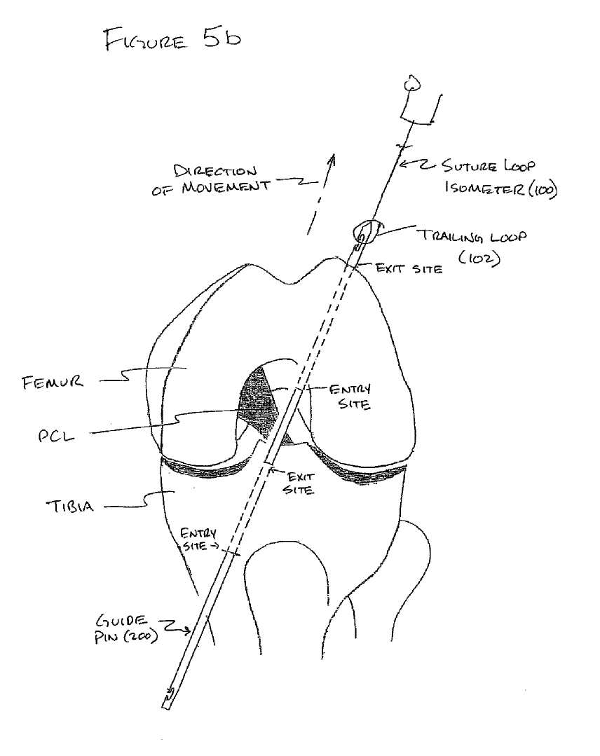

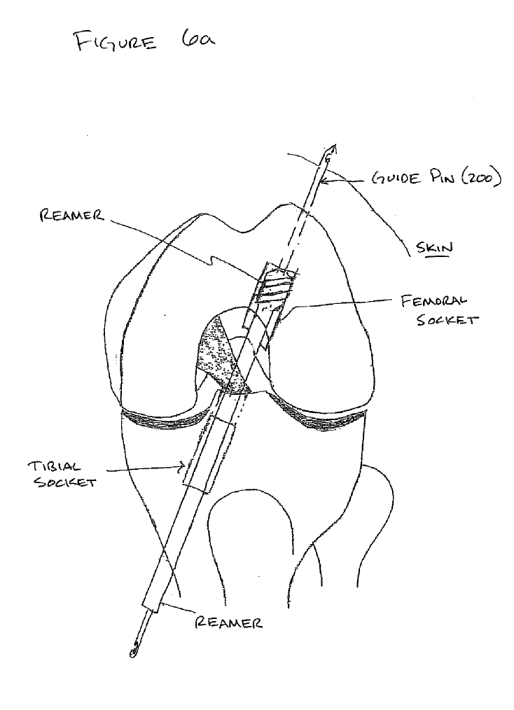

FIGS. 3-6b illustrate embodiments of a trans-tibial ACL approach according to the present invention. In one embodiment, the proposed trans-tibial approach includes placing the double slotted guide pin into the tibia, across the joint, and into the femur without reaming any holes (FIG. 3). In one embodiment, the suture loop isometer is passed, in the direction of movement illustrated, across the joint with both the leading loop and the free end attached to the distal end of the guide pin (FIG. 3). In one embodiment, the leading loop is passed ahead of the free end so that the “T-bar” portion of the suture loop isometer is in a position which is generally parallel with the elongated body of the suture loop isometer, thereby allowing passage of the device through the tibial and femoral holes created by the guide pin (FIG. 4). In one embodiment, the T-bar is sized to correspond to the diameter of the holes after the femur and tibia are reamed. In one embodiment, the elongated body of the suture loop isometer is sufficient to allow the T-bar portion to extend through the exit site of the femur while the trailing loop remains outside the entry side of the tibia (FIG. 5a). In one embodiment, once the T-bar portion of the suture loop isometer has exited the femur during passage, the free end may be pulled to make the T-bar perpendicular to the femoral cortex and drill hole (FIG. 5a). In one embodiment, the direction of movement of the suture loop isometer is reversed and the trailing loop end is pulled forcing the T-bar portion against the femoral cortex superiorly to allow for simulation of an ACL graft. In one embodiment, the pull may be done manually while the knee is cycled through flexion and extension. In one embodiment, if the suture appears to piston in and out of the guide pin hole during cycling of the knee, a new guide pin hole is drilled and the process repeated to achieve isometricity. In one embodiment, once isometricity is achieved, the suture loop isometer is advanced farther through the femur in a retrograde maneuver with the guide pin attached on the trailing loop end of the suture loop isometer (FIG. 5b). This allows passage of the pin through the exact pathway that the isometric suture loop has traveled (FIG. 5b). In one embodiment, a tibial and femoral reamer is then passed over the guide pin to create ACL sockets in a standard fashion (FIG. 6a). In one embodiment, the reamer is then removed and the double slotted guide pin is advanced once again superior and laterally out of the skin with the ACL graft attached (FIG. 6b). In one embodiment, the ACL graft is then positioned in place and standard fixation is performed to complete the procedure.

### B. Medial Portal Approach

ACL surgery may be performed through a medial approach in which the femoral tunnel is addressed prior to the tibial tunnel. The basic difference between this technique and the trans-tibial approach is that the tunnels are created independent of each other.

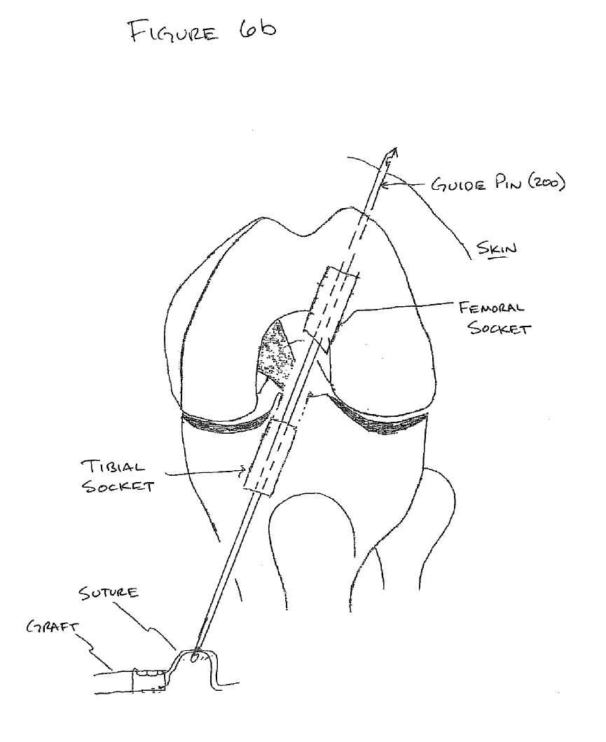

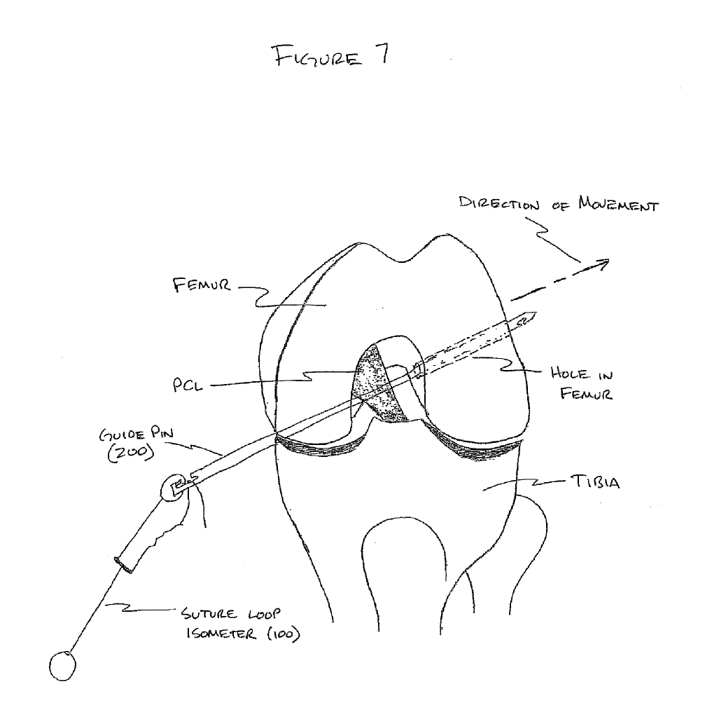

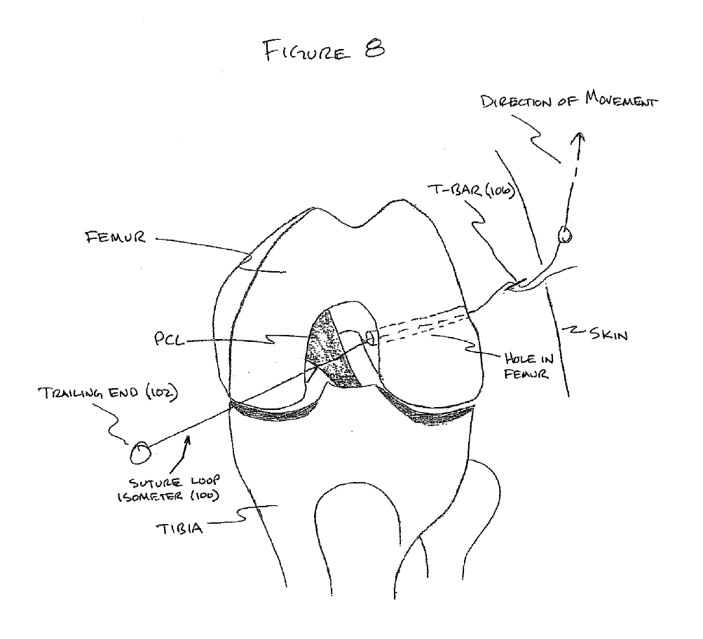

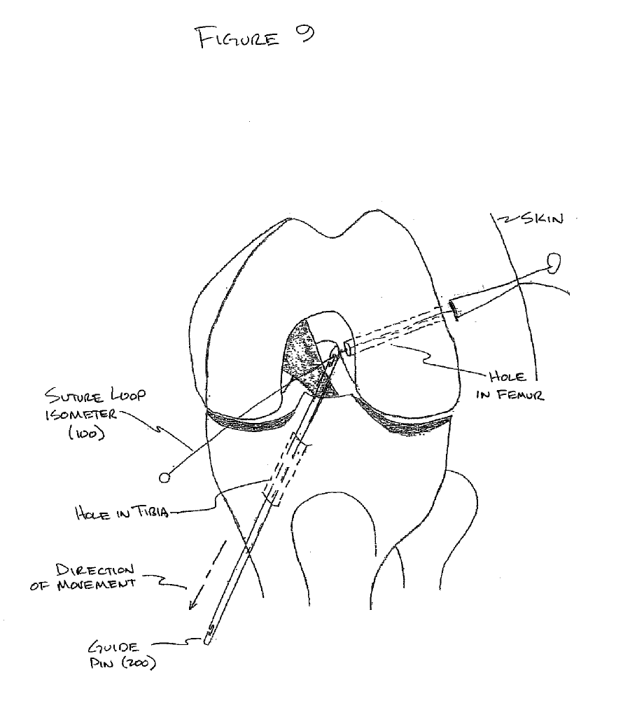

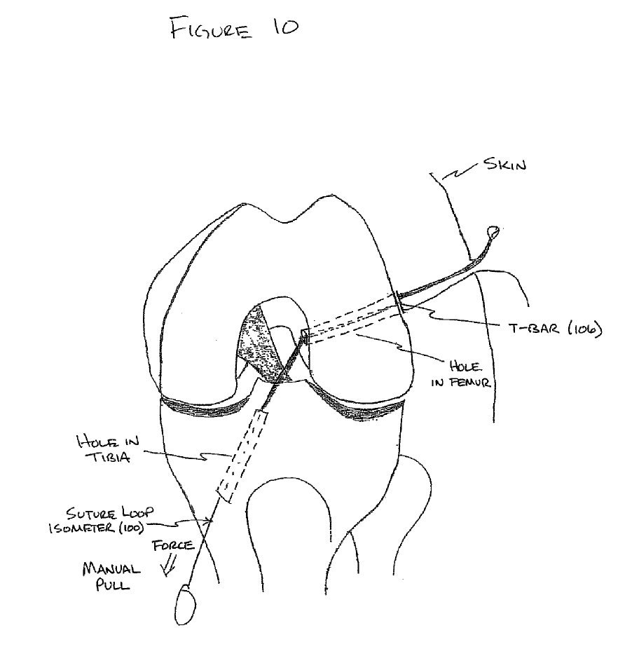

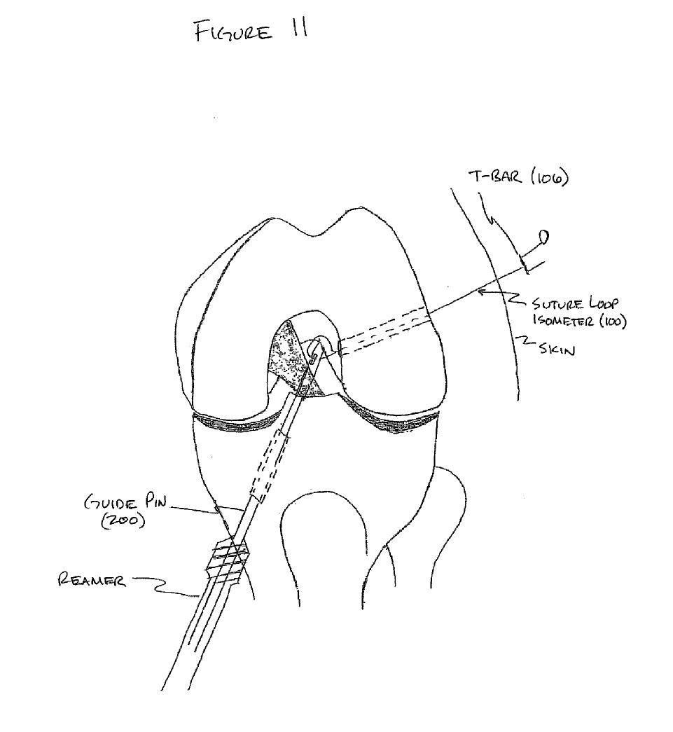

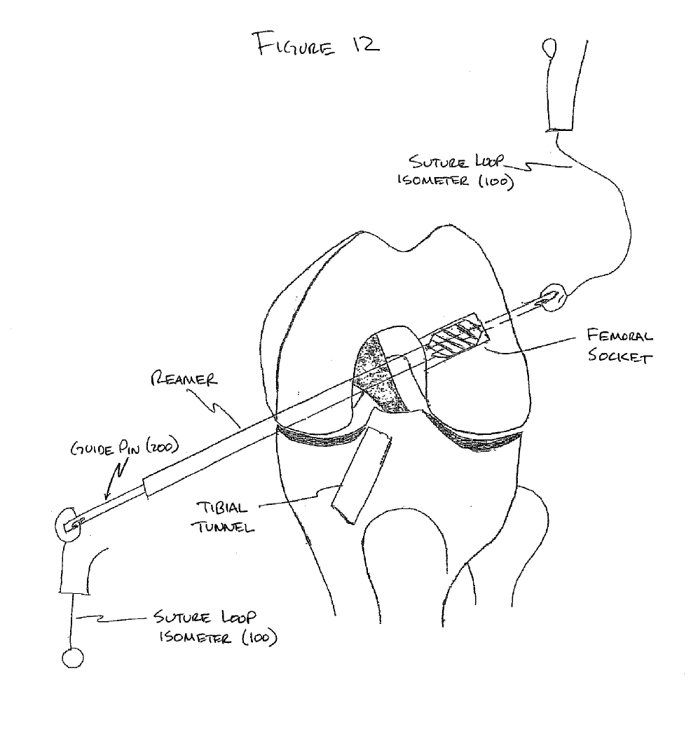

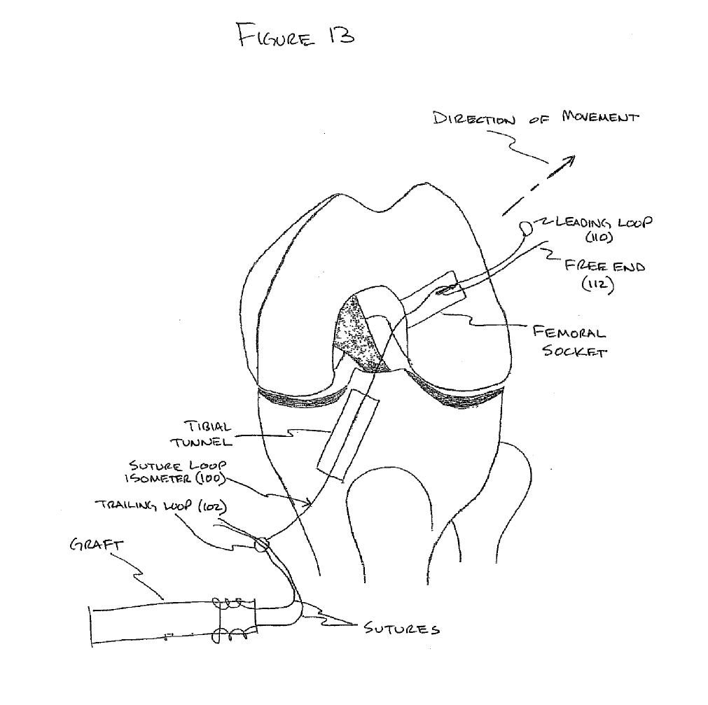

FIGS. 7-13 illustrate embodiments of a medial portal approach according to the present invention. In one embodiment of this technique, the guide pin is placed through the medial portal and into the femur with the leading loop and free end attached to the distal end of the guide pin (FIG. 7). In one embodiment, similar to the trans-tibial approach, the leading loop is passed in the direction of movement illustrated first to facilitate passage of the T-bar portion through the drill hole in the position generally parallel with the elongated body and the holes (FIG. 8). In one embodiment, a standard ACL guide may be used to place the guide pin through the tibia and into the joint while the suture loop remains in position in the femur (FIG. 9). In one embodiment, the elongated slot at the proximal end of the guide pin captures the suture loop and then the guide pin is removed from the tibia (FIG. 9). In one embodiment, while the suture loop isometer is in place with force causing resistance of the T-bar portion against the femur, the knee is cycled with flexion and extension to assess isometricity (FIG. 10). Once isometricity is achieved, the guide pin is re-passed back across the tibia using the trailing loop to guide the pin into position (FIG. 11). In one embodiment, the tibial tunnel is created using a reamer passed over the guide pin (FIG. 11). The trailing loop of the suture loop isometer is then retrieved out of the medial portal and attached to the leading end of the guide pin to facilitate passage of the guide pin across the femur (FIG. 12). In one embodiment, with the guide pin in place, the femoral reamer is passed over the guide pin to create the femoral socket (FIG. 12). The reamer is removed and the guide pin is then advanced completely through the femur with the suture loop isometer attached once again. In one embodiment, the trailing loop of the suture loop isometer is then retrieved through the tibial tunnel in order to recreate the pathway of the ACL prior to passage of the graft (FIG. 13). In one embodiment, the graft is then passed through the tibia, across the joint, and into the femur in standard fashion.

### C. Double Bundle ACL

Double bundle ACL reconstruction is another alternative technique for addressing an ACL tear. Using this technique, instead of reconstructing one large bundle, as discussed above, two independent smaller bundles are created called the AM (anteromedial) and PL (posterolateral) bundles.

Despite the tunnels being considerably smaller in size than a single bundle technique, the amount of space required to accommodate the two tunnels in the femur and tibia is more than the single bundle technique. Furthermore, with four tunnels being drilled (two in the femur and two in the tibia) there is more room for error with regards to isometricity. Assessing whether or not an adequate bone bridge between the two tunnels is available is paramount. Having the two tunnels converge or fracture into one another can be catastrophic for the proposed procedure. Therefore, determining whether or not there is enough native tibial/femoral footprint (size) available to successfully complete a double bundle ACL reconstruction prior to committing to this technique would be helpful.

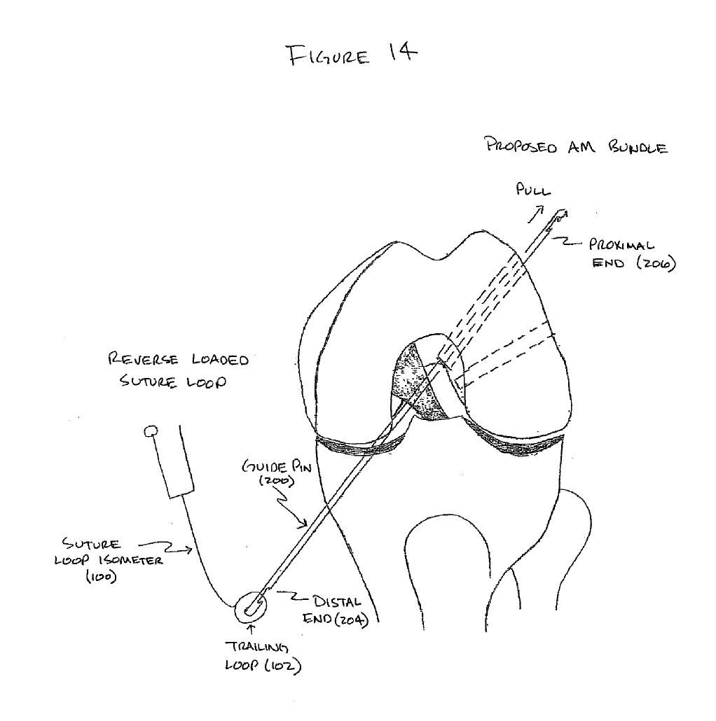

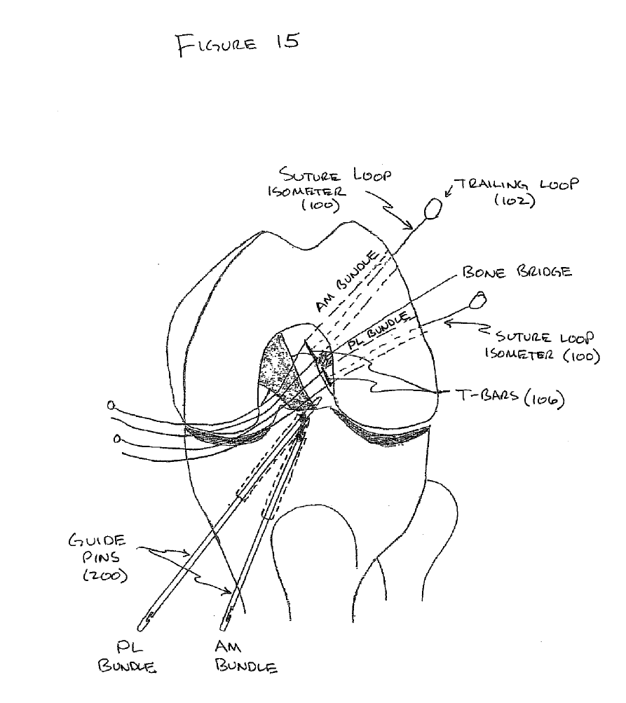

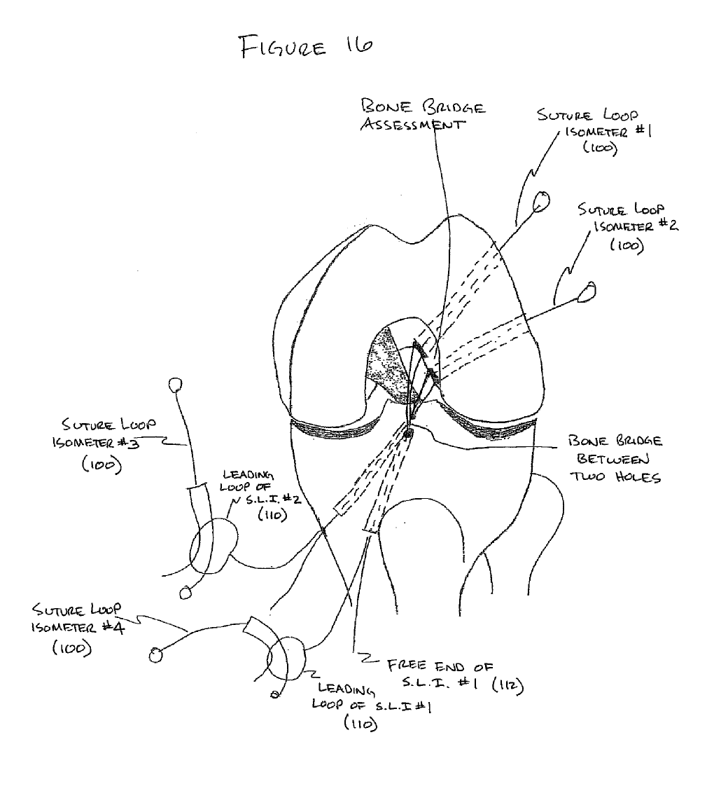

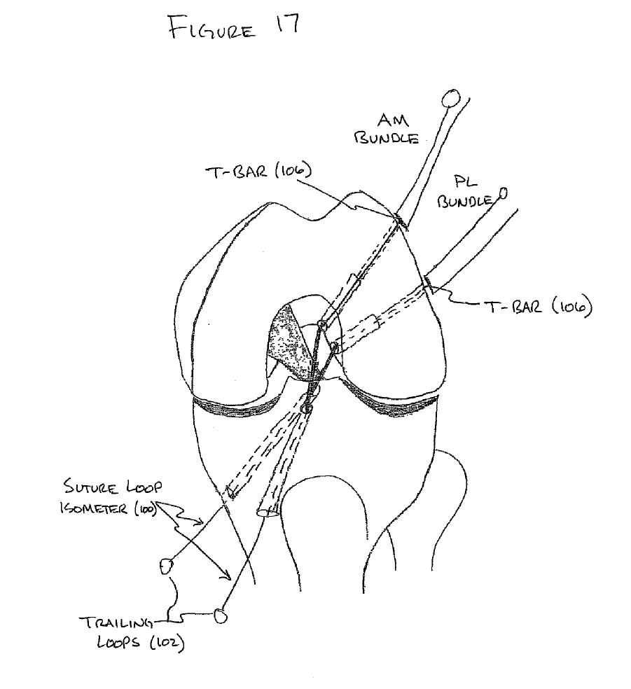

FIGS. 14-22 illustrate embodiments of a double bundle approach according to the present invention. FIG. 14 illustrates installing a guide pin through the medial portal into the proposed AM bundle location, according to one embodiment. In one embodiment, this is done using a standard ACL offset guide. In one embodiment, the guide pin is pulled through the femur with the trailing end of the suture loop isometer attached to the distal end of guide pin (FIG. 14). In one embodiment, the PL bundle is then performed in a similar fashion (FIG. 15). In one embodiment, once the reverse loaded suture loops are in place on the native ACL footprint, the proposed tunnels may be checked for location and size. In one embodiment, the T-bar portions of the suture loop isometers are available in a suitable width to exactly replicate the individual bundle diameters. In one embodiment, the distance between the T-bar portions is assessed to determine if the bone bridge is adequate (FIG. 15). In one embodiment, guide pins are then brought up through the AM and PL bundle locations in the tibia. In one embodiment, the free end and leading loop of suture loop isometers #1 and #2, respectively, are attached onto each respective guide pin and delivered out of the tibia (FIGS. 15 and 16). Additional suture loop isometers #3 and #4, respectively, may be attached in reverse (i.e., leading loop and free end of the suture loop isometer #4 attached to leading loop of suture loop isometer #1) and pulled up through the tibia and femur to achieve fully spanned suture loops across each of the bundle pathways and checked for isometricity (FIG. 17). In one embodiment, guide pins are then placed and reamers are passed to create sockets for the ACL bundles.

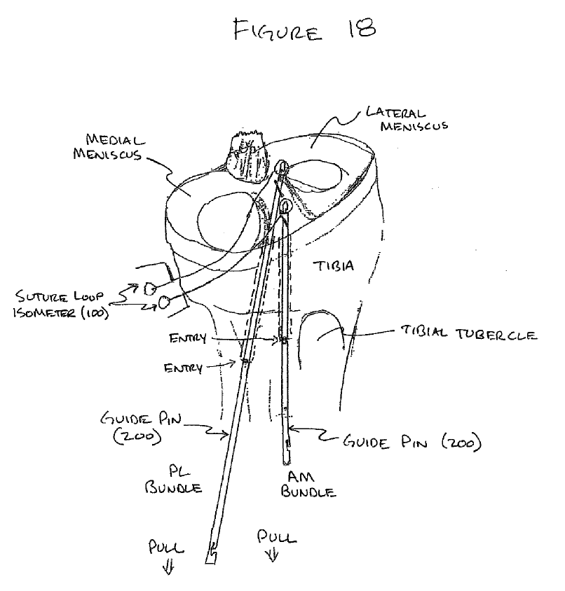

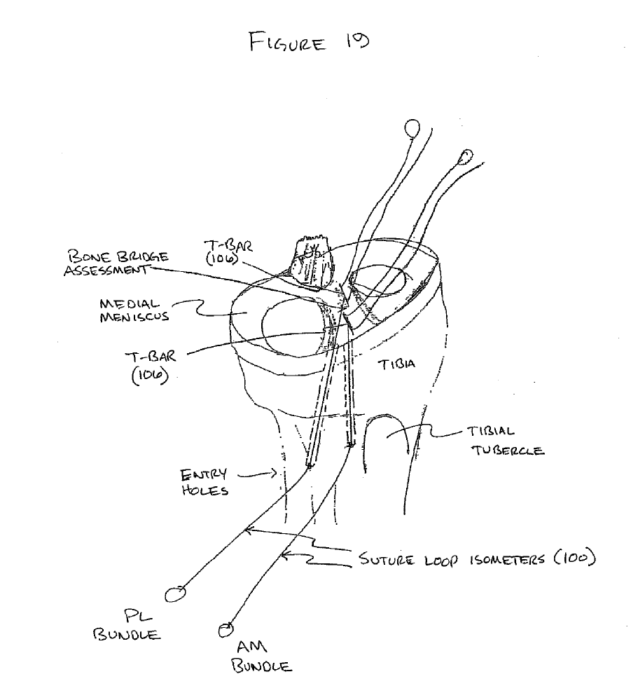

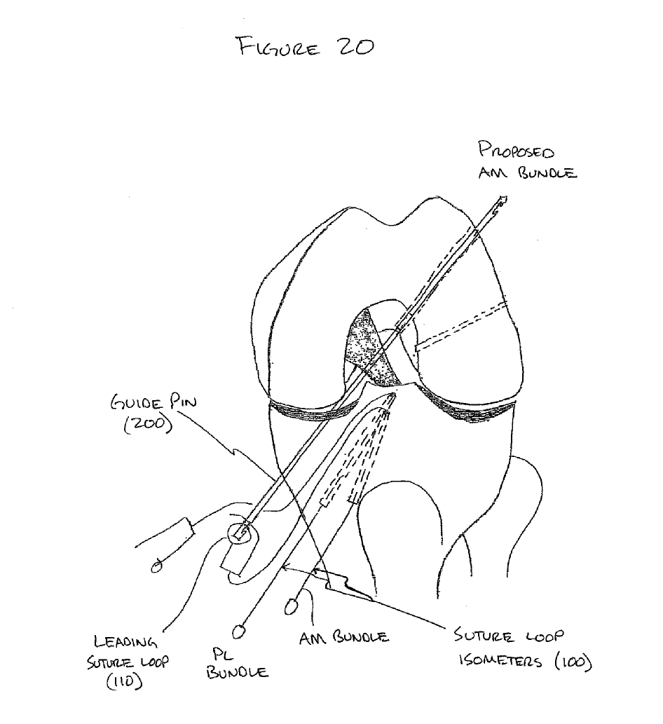

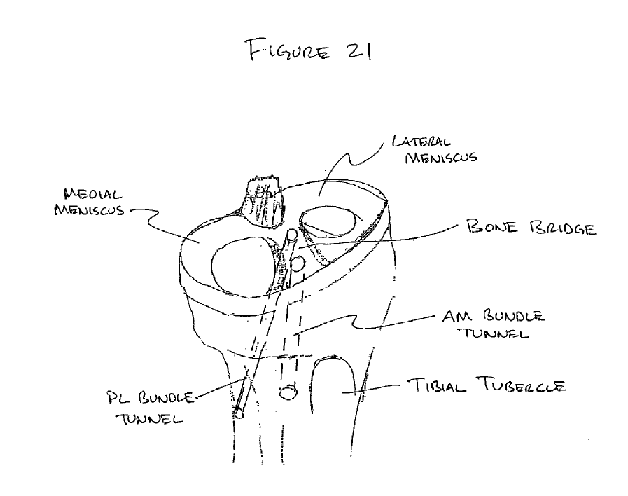

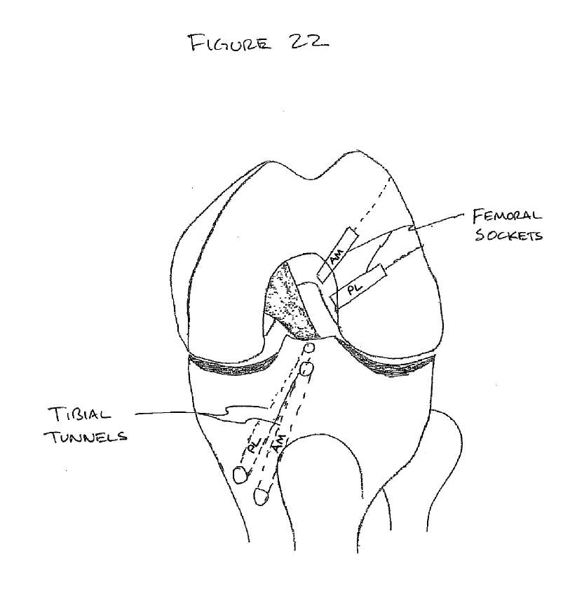

Alternatively, as illustrated in FIGS. 18-22, the tibia is accessed first when performing the double bundle reconstruction. The lack of anterior to posterior distance on the native tibial ACL footprint is more commonly encountered than in the femoral footprint. In one embodiment of this technique, the AM and PL footprint guide pins are placed. In one embodiment, the trailing ends of suture loops are introduced with a grasper through the medial portal and hooked onto the proximal end of the guide pins (FIG. 18). In one embodiment, the guide pins are then pulled back out of the tibia to allow the T-bar portions to sit flush on the tibial footprint (FIG. 19). The bone bridge between the T-bar portions is then assessed (FIG. 19). In one embodiment, the femoral guide pins are then placed sequentially by passing each pin through the medial portal with their respective leading suture loop attached to the distal end of the guide pin (FIG. 20). In one embodiment, final isometricity can then be checked with each suture bundle in place. In one embodiment, guide pins are then placed sequentially and reamers are passed to create sockets for the ACL bundles (FIGS. 21 and 22).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### REFERENCE NUMERALS IN THE FIGURES ARE IDENTIFIED AS FOLLOWS

100—suture loop isometer
102—trailing loop
104—elongated body
106—T-bar
108—leg
110—leading loop
112—free end
200—guide pin
202—slotted opening
204—distal end
206—proximal end
208—constricted or necked opening
210—body
212—exterior surface
214—pointed tip

What is claimed is:

1. A suture loop isometer, comprising:
an elongated body;
a first loop provided at one end of the elongated body;
a T-bar provided at an opposite end of the elongated body;
at least one leg extended from one end of the T-bar; and
a second loop provided at an end of the at least one leg.

2. The suture loop isometer of claim 1, wherein the suture loop isometer is formed of absorbable suture material.

3. The suture loop isometer of claim 1, wherein the elongated body, the first loop, the at least one leg, and the second loop are all flexible.

4. The suture loop isometer of claim 3, wherein the T-bar is less flexible than the elongated body and the at least one leg.

5. The suture loop isometer of claim 1, further comprising:
another leg extended from another end of the T-bar.

6. The suture loop isometer of claim 5, wherein the at least one leg and the another leg freely extend from opposite ends of the T-bar.

7. The suture loop isometer of claim 6, wherein the at least one leg and the another leg extend substantially parallel with each other in a direction away from the elongated body.

8. The suture loop isometer of claim 1, wherein the T-bar is connected to the elongated body at approximately a midpoint of the T-bar.

9. The suture loop isometer of claim 1, wherein the T-bar is freely pivotable about the opposite end of the elongated body.

10. The suture loop isometer of claim 9, wherein, in a first position, the T-bar is oriented substantially perpendicular to the elongated body.

11. The suture loop isometer of claim 10, wherein, in a second position, the T-bar is oriented substantially parallel with the elongated body.

12. A suture loop isometer, comprising:
an elongated body having a first end and a second end;
a trailing loop provided at the first end of the elongated body;
a T-bar freely pivotably connected to the second end of the elongated body;
a first leg extended from a first end of the T-bar;
a leading loop provided at an end of the first leg; and
a second leg extended from a second end of the T-bar.

13. The suture loop isometer of claim 12, wherein the suture loop isometer is formed of absorbable suture material.

14. The suture loop isometer of claim 12, wherein the elongated body, the trailing loop, the first leg, the leading loop, and the second leg are all flexible, and the T-bar is less flexible than the elongated body, the first leg, and the second leg.

15. The suture loop isometer of claim 12, wherein the T-bar is connected to the second end of the elongated body at approximately a midpoint of the T-bar, and wherein the first leg and the second leg extend substantially parallel with each other in a direction away from the elongated body.

16. The suture loop isometer of claim 12, wherein the T-bar is pivotable between a first position oriented substantially perpendicular to the elongated body, and a second position oriented substantially parallel with the elongated body.

17. An isometer system, comprising:
a guide pin having a distal end, a proximal end, and a linear body extending between the distal end and the proximal end, wherein the distal end includes a first slotted opening, and wherein the proximal end includes a second slotted opening and a pointed tip; and
a suture loop isometer having an elongated body, a trailing loop provided at one end of the elongated body, a T-bar provided at an opposite end of the elongated body, a first leg extended from one end of the T-bar, a leading loop provided at an end of the first leg, and a second leg extended from an opposite end of the T-bar,
wherein the first and second slotted openings of the guide pin are configured to selectively accept the leading and trailing loops of the suture loop isometer.

18. The system of claim 17, wherein the guide pin is substantially rigid and the suture loop isometer is substantially flexible.

19. The system of claim 17, wherein the first and second slotted openings of the guide pin each have a constricted opening at an exterior surface of the guide pin.

20. The system of claim 17, wherein the T-bar of the suture loop isometer is freely pivotable about the opposite end of the elongated body, and wherein the first and second legs of the suture loop isometer each freely extend from the T-bar away from the elongated body.
